(19) United States
(12) Patent Application Publication
(10) Pub. No.: US 2025/0161170 A1
(43) Pub. Date: May 22, 2025

(54) LIP STAIN PRODUCT AND METHOD OF APPLICATION

(71) Applicant: Be The Envy LLC, Sorrento, FL (US)

(72) Inventor: Yolanda HALSTON, Sorrento, FL (US)

(21) Appl. No.: 18/951,028

(22) Filed: Nov. 18, 2024

Related U.S. Application Data
(60) Provisional application No. 63/599,690, filed on Nov. 16, 2023.

(51) Int. Cl. A61K 8/02; A61K 8/34; A61K 8/60; A61Q 1/04

(52) U.S. Cl. CPC A61K 8/0208; A61K 8/345; A61K 8/608; A61Q 1/04; A61K 2800/432

(57) ABSTRACT

A lip stain product applicable to and absorbable by a user's lips. The lip stain product includes a substrate having a first side and a second side and a lip stain comprised of ink, the lip stain at least one of imprinted and embedded on at least the first side of the substrate, wherein when at least one of the user's lips and the ink is moistened, the lip stain transfers directly to the lips of the user.

### CROSS-REFERENCE TO RELATED APPLICATION

This application is related to and claims priority to U.S. Provisional Patent Application Ser. No. 63/599,690, filed Nov. 16, 2023, entitled LIP STAIN PRODUCT AND METHOD OF APPLICATION, the entirety of which is incorporated herein by reference.

### TECHNICAL FIELD

The present disclosure relates to the field of lip stains and more specifically to a lip stain preparation and method of applying the preparation to a user's lips, as well as a method of allowing users to print out their own lip stain colors and/or designs via access to a software application.

### BACKGROUND

For many years, lipstick has be used as a cosmetic for enhancing or altering the color of the lips. Lipstick is a cosmetic color in a wax carrier and has the disadvantage of being easily transferrable from the lips to other objects. Lipstick smears and rubs off while drinking, kissing or by any other contact of the lips with other articles such as coffee cups, tissue, and clothing or simply by licking the lips. This leads to the loss of an even and uniform application, which requires frequent reapplication. Attempts have been made to provide an alternative color cosmetic to the lips which do not have the disadvantages of lipstick, however these tend to cause dryness and cracking.

Lip stain or lip tint is a cosmetic product used to color the lips, usually in the form of a cream, liquid or gel. While lip stain might look similar to lip gloss or lipstick, the difference is that typical lip stain is formulated so that the color is absorbed by the user's lips, rather than just lying on the lips like lipstick. With lip stain, the color lasts longer and needs less touching up. Lip stain last longer than lipstick because of its ability to stain the lips instead of just covering and coating them with color.

However, there are drawbacks with typical lip stains and lip stain preparations and applications. In typical lip stain application, the lip stain, usually in the form of a cream or liquid in a bottle, is applied to the lips with an applicator wand, felt tip marker, or brush. This procedure is time-consuming and relies on the user to apply a steady hand when applying the lip stain, resulting in hard-to-fix mistakes. In many instances, the wand tip or brush tends to dry out. Further, the bottle of cream or liquid may spill and/or leak. Also, many lip stains include high levels of alcohol, which tends to dry the lips. In addition, users are limited in the availability and accessibility to various lip stain colors and designs.

The present disclosure addresses and improves upon the aforementioned drawbacks.

### SUMMARY

Described herein is a lip stain product applicable to and absorbable by a user's lips. In accordance with one aspect of the present disclosure, the lip stain product includes a substrate having a first side and a second side, and a lip stain comprised of ink, the lip stain at least one of imprinted and embedded on at least the first side of the substrate, wherein when at least one of the user's lips and the ink is moistened, the lip stain transfers directly to the lips of the user.

In one embodiment of this aspect, the lip stain product is produced on a printer using CMYK cartridges filled with ink. In another embodiment, the ink can be a blended ink of various colors and shades, which allows any specified color to be used in the actual manufacture/printing of the lip stain product that will be used. In another embodiment, the ink is edible. In another embodiment, the substrate is soft tissue paper. In another embodiment, the substrate is wax paper.

In another embodiment, the lip stain covers less than 50% of a surface area of the first side of the substrate. In another embodiment, the lip stain covers more than 50% of a surface area of the first side of the substrate. In another embodiment, the lip stain covers at least 80% of a surface area of the first side of the substrate. In another embodiment, the substrate further comprises alignment indicia indicating where the substrate is to be folded prior to transferring the lip stain to the user's lips.

In another embodiment, the lip stain comprises less than all of the first side of the substrate thus leaving unstained portions of the first side of the substrate. In another embodiment, the unstained portions comprise a first handling area and a second handling area allowing the user to grasp each of the first and second handling areas before and while applying the lip stain to the user's lips.

In another embodiment, the ink comprises water, and at least one of Propylene Glycol, Glycerin, Poly Sorbate 80, and Mono and Di-Glycerides. In another embodiment, the ink further comprises at least one of FD&C Red No. 40, FD&C Blue No. 1, FD&C Yellow No. 5, and FD&C Yellow No. 6.

In accordance with another aspect of the present disclosure, a method of transferring lip stain from a lip stain product substrate to a user's lips is provided. In one embodiment of this aspect, the method includes arranging the substrate such that the lip stain faces the user's lips, moistening at least one of the lip stain and the user's lips, and transferring the lip stain to the user's lips when he lip stain makes contact with the user's lips.

In one embodiment of this aspect, the method further includes folding the lip stain product substrate in accordance with alignment indicia located on the lip stain product substrate prior to transferring the lip stain to the user's lips. In another embodiment, the lip stain includes ink, wherein the ink is at least one of non-alcoholic, edible and vegan.

In another embodiment, the method further includes folding the substrate such that two surfaces of the lip stain are exposed. In another embodiment, the method further includes providing alignment indicia indicating where the substrate is to be folded prior to transferring the lip stain to the user's lips.

In accordance with another aspect of the present disclosure, a computer-implemented method for allowing a user to create one or more lip stains is provided. The method includes receiving, at a centralized server, user identification information identifying a user, receiving, at the centralized server, a selection by the user of at least one lip stain, the selection including at least one of lip stain color and shade, and providing the user with access to the selected lip stain and with the ability to print out the selected lip stain on a lip stain product substrate. In accordance with one embodiment of this aspect, the method further includes providing a template populated with a variety of colors, shades, sizes and dimensions, wherein the user can select one or more lip stains from the template.

### BRIEF DESCRIPTION OF THE DRAWINGS

FIG. 1A illustrates an exemplary lip stain product of the present disclosure;

FIG. 1B illustrates the lip stain product of FIG. 1A with different lip stain dimensions;

FIG. 1C illustrates the lip stain product of FIG. 1A with still different lip stain dimensions;

FIG. 1D illustrates the lip stain product of FIG. 1A with still further different lip stain dimensions;

FIG. 2 illustrates how the lip stain product of the present disclosure is folded before application;

FIG. 3 illustrates how the lip stain is transferred to a user's lips; FIG. 1 is a block diagram of an example system according to some embodiments of the present disclosure;

FIG. 4 is a block diagram of an exemplary computer-based system according to some embodiments of the present disclosure; and

FIG. 5 is a block diagram of an example computing environment of FIG. 4 according to some embodiments of the present disclosure.

### DETAILED DESCRIPTION

The present disclosure is both a lip stain product and a method of applying a lip stain product to a user's lips. The lip stain product of the present disclosure is transfer-proof, smear-proof and waterproof. It provides the perfect solution for busy women to wear for weddings, for use at the beach, gym, and all active sports. The wearer can swim, shower and sleep while wearing the disclosed lip stain, since it will not smear or fade. The appearance can be further enhanced by applying other types of lip product such as lipstick and lip gloss over the top of lip stain of the present disclosure as it will not fade or transfer as the other products wear off, thus providing long lasting continuous color that will last all day.

In one embodiment, the lip stain product disclosed herein is made of edible ink formula that can create shades of virtually every Pantone color. In one embodiment, the ink is non-alcoholic. In one embodiment, the lip stain is produced using printers with CMYK cartridges filled with edible, vegan ink. In one embodiment, the ink is pre-applied to a medium or “substrate” such as soft tissue paper, although other media may also be used. In one embodiment, the ink is applied to/printed on dry wax paper in order to limit the absorption of the ink into the paper and allow for easy transfer to the user's lips. Other substrates may be used, including, for example, card stock or paper of varying weight and thickness. In one embodiment, using this approach, the user can select the shade of lip stain they want, and purchase boxes of lip stain in their selected color/shade, with ink already pre-applied to the substrate, and ready to be applied to the user's lips. After wetting their lips and/or wetting the paper and transferring color to their lips with the lip stain product, the user's lips exhibit a smooth, buildable finish.

In another embodiment, the lip stain product can be assembled by the user with a custom handheld or desktop printer, substrate and ink cartridges where the consumer can print their own lip stain product. This embodiment is discussed in further detail below.

In yet another embodiment, a software application downloaded to the user's computer or mobile device, or made available on a third party website, can be utilized to allow the user to select different colors and/or shades of lip stain, and to select a printer where the lip stain product can be printed. This embodiment is discussed in further detail below.

Disclosed herein is also a method of applying the lip stain product of the present disclosure to a user's lips. In one embodiment, the user folds the paper upon which the lip stain is embedded and or imprinted lengthwise with the colored lip stain facing out. The user then moistens their lips and/or moistens the lip stain and presses their lips on the paper upon which the colored lip stain is transferred to the user's lips.

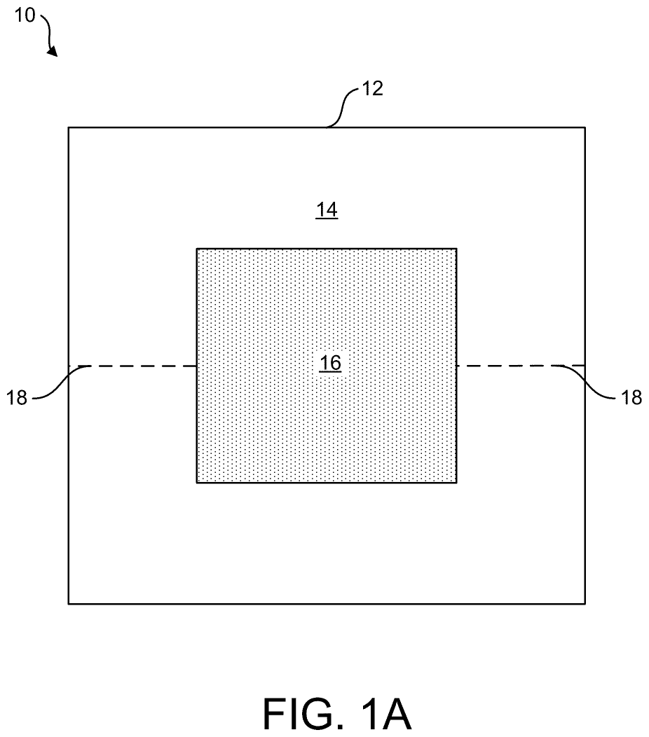

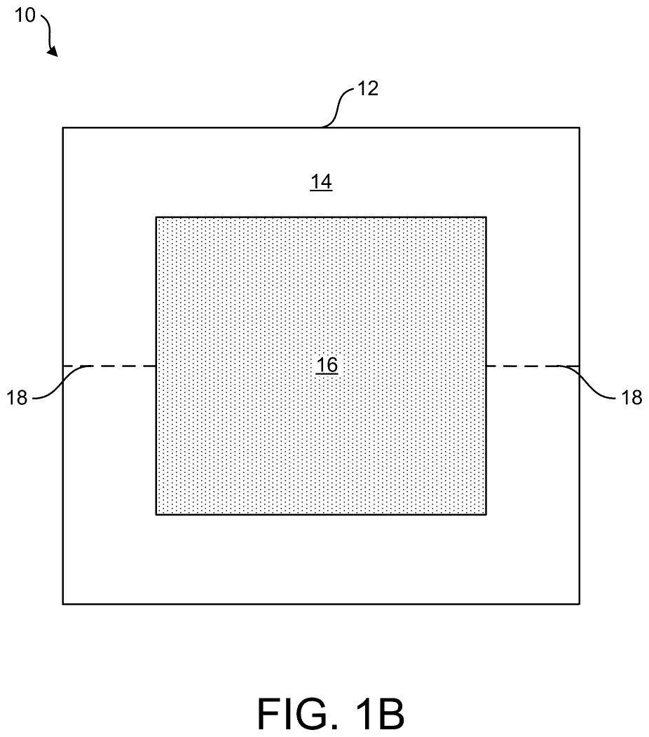

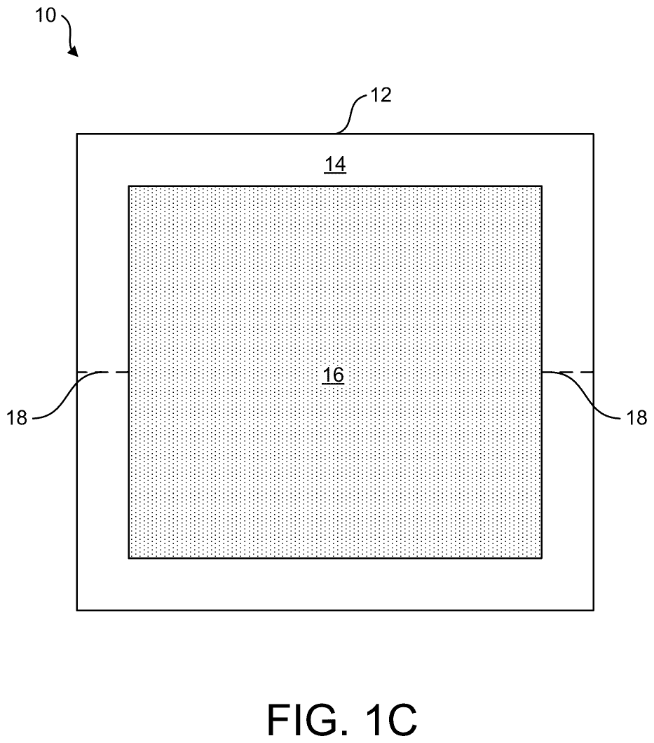

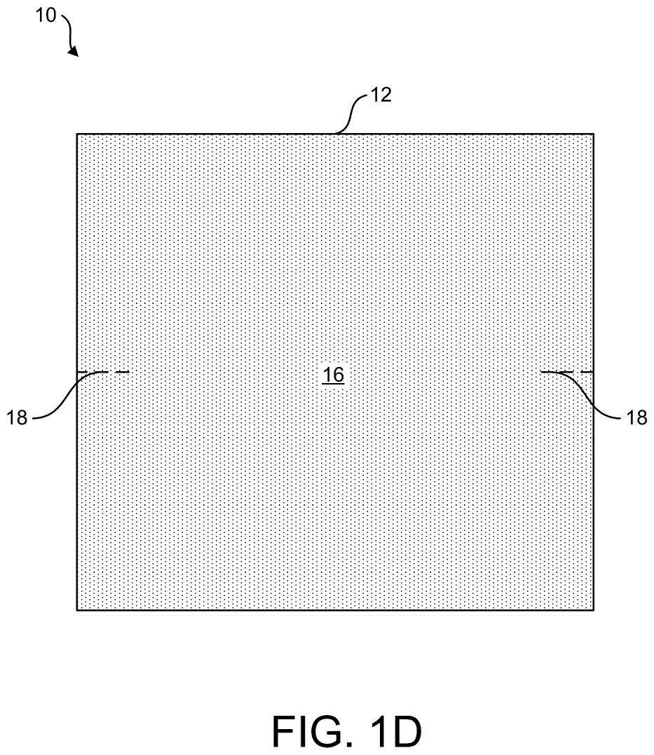

Referring now to the FIGS. 1A-1D, exemplary lip stain products of the present disclosure are shown. In FIG. 1A the lip stain product 10 of the present disclosure is shown. Lip stain product 10 includes an outer medium or substrate 12, which can be comprised of various types of materials in various sizes, shapes, and dimensions. Although substrate 12 is shown as a square in FIG. 1A, it is within the scope of the present disclosure to provide a lip stain product that includes a substrate 12 of any shape. For example, substrate 12 could be a rectangle, circle, or any other shape. Further, substrate 12 could be of any size. For example, in one non-limiting embodiment, substrate 12 can be the size of an average business card, 2″×3 ½″, or square shaped, i.e., 3″×3″. Again, these dimensions and shapes are exemplary only.

Substrate 12 includes a first side 14 and a second side (not shown). Substrate 12 can be comprised of different types of materials, including, for example, soft tissue paper, dry wax paper, or a “Post-It” style paper, i.e., paper with adhesive on the second side of substrate 12. Other types of substrates may be used including card stock or paper of varying weights and thickness.

Embedded or imprinted on first side 14 of substrate 12 is a colored lip stain 16. In one non-limiting embodiment, the ink comprising lip stain 16 includes water, and any combination of Propylene Glycol, Glycerin, Poly Sorbate 80, Mono and Di-Glycerides, and may also contain any combination of Allura Red AC, Brilliant Blue FCF, Tartrazine, and Sunset Yellow FCF. In another non-limiting embodiment, the ink comprising the lip stain 16 includes water, and any combination of Propylene Glycol, Glycerin, Poly Sorbate 80, and Mono and Di-Glycerides, and may also contain any combination of FD&C Red No. 40, FD&C Blue No. 1, FD&C Yellow No. 5, and FD&C Yellow No. 6. In yet another non-limiting embodiment, lip stain 16 does not include Erythrosine or Red #3. The afore-mentioned examples are not meant to be limiting in any way.

Lip stain 16 can be of varying colors and shades of the user's choosing. Although FIG. 1A shows lip statin 16 located on the first side 14 of substrate 12 in a square shape, it is within the scope of the present disclosure to provide a lip stain 16 in any shape. For example, lip stain 16 could be a rectangle, circle, or any other shape. Further, lip stain 16 could be of any size. Lip stain 16 includes ink that when wet, and/or applied to the user's moistened lips, transfers smoothly from substrate 12 to the user's lips.

While FIG. 1A shows lip stain 16 in the substantial center of the first side 14 of substrate 12, the present disclosure is not limited in this way, and lip stain 16 may be off-center, etc. However, by placing lip stain 16 in the substantial center of the first side 14 of substrate 12, it is easier for the user to align their lips to the lip stain 16 prior to transfer of the colored ink comprising lip stain 16 to the user's lips.

FIGS. 1B and 1C show a lip stain product 10 similar to the lip stain product 10 shown in FIG. 1A, except that in FIGS. 1B and 1C, lip stain 16 is larger and thus takes up more surface area of the first side 14 of substrate 12. Thus, it is within the scope of the present disclosure to provide a lip stain product 10 where lip stain 16 takes up a smaller portion, i.e., 50% or less, or a larger portion, i.e., between 50% and 80%, of substrate 12. These percentages are exemplary only, and thus lip stain 16 can take up any percentage of the first side 14 of substrate 12. When applying the lip stain 16, the user can place their hands on the portion of first side 14 of substrate 12 that does not have ink from lip stain 16 on it so their hands will remain ink-free. Thus, if less than 100% of the surface of the first side 14 of substrate 12 contains lip stain 16, the remaining portions are essentially “unstained” and in some embodiments, comprise a first handling area and a second handling area allowing the user to, advantageously, grasp each of the first and second handling areas before and while applying the lip stain 16 to the user's lips.

In some instances, lip stain 16 can cover the entire surface of substrate 12, i.e., from edge-to-edge. This embodiment is shown in FIG. 1D. In this embodiment, lip stain 16 is flood-coated from edge-to-edge of the first side 14 of substrate 12. Advantageously, the lip stain product 10 of the present disclosure allows the user to select not only the different colors and shades of lip stain 16 (discussed in further detail below), but also allows the user to select different sized substrates 12 with different sizes and shapes of lip stain 16. This is advantageous because user's faces are shaped differently. For example, adults may select a lip stain product 10 with a substrate 12 that is larger, and/or has a larger amount of lip stain 16 disposed on the first side 14 of substrate 12, while a teenager or younger person may select a smaller substrate 12 and smaller amount of lip stain.

FIGS. 1A-1D also include an embodiment where alignment indicia 18 are disposed upon substrate 12. In one embodiment, alignment indicia 18 could be in the form of one or more lines, which provide the user with a guideline along which to fold substrate 12 prior to transferring the ink of lip stain 16 to the user's lips. The present disclosure is not limited in any way to the type, shape, size or dimensions of alignment indicia 18. As will be shown in the ensuing figures and discussed in further detail below, prior to application, the user folds substrate 12 along the lines presented by alignment indicia 18, which, after folding, results in exposing the ink of lip stain 16 on two opposite and exposed surfaces, where the two surfaces are in fact the first side 14 of substrate 12. The folding of substrate 12 to provide lip stain 16 on two surfaces advantageously make it easier for the user to apply their lips to the lip stain 16, which allows the ink of lip stain 16 to be transferred to the upper and lower lips of the user.

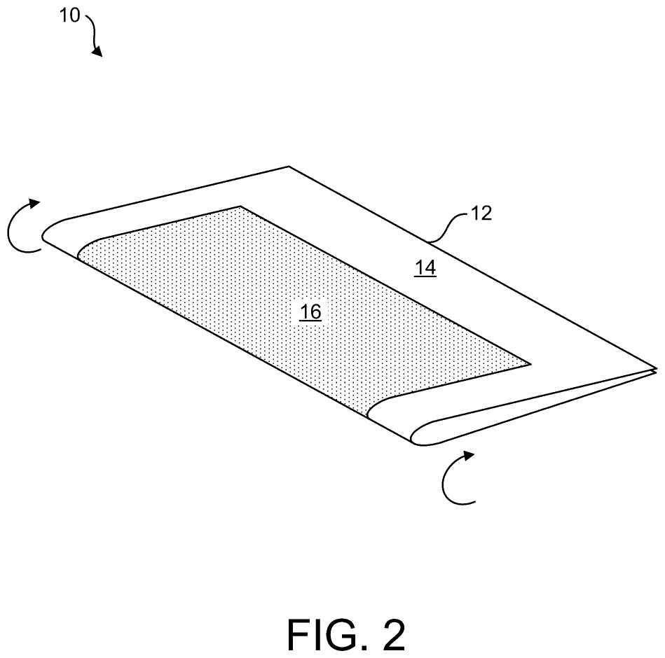

FIG. 2 shown an exemplary lip stain product 10 folded in the direction of the arrows along alignment indicia 18 (not shown in FIG. 2). In FIG. 2, the user simply folds substrate 12 along the alignment indicia 18 to expose lip stain 16 on both the first side 14 and the second side (not shown) of substrate 12. Thus, what is shown in FIG. 2 is the result of lip stain product 10 after the user has folded it. The purpose of folding the substrate 12 in the manner shown is to provide lip stain 16 on two surfaces of first side 14, which will enable the user to simply touch their lips to the exposed two surfaces containing the lip stain 16, resulting in lip stain 16 applied evenly to the user's upper and lower lips.

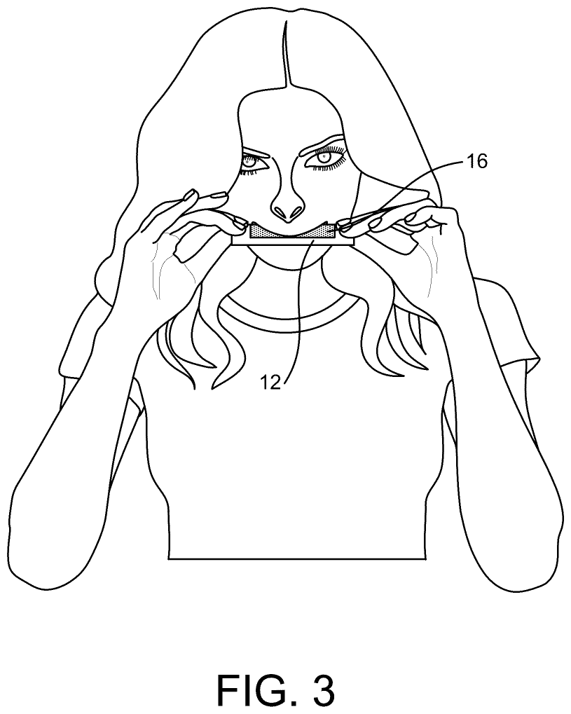

Referring now to FIG. 3, the lip stain product 10 of the present disclosure is shown in use. After the user has folded substrate 12 as shown in FIG. 2, with (or without) the use of alignment indica 18, the lip stain 16 is now exposed on opposing surfaces of the first side 14 of substrate 12. Prior to use, the lip stain 16 is moistened either by dipping substrate 12 in water or applying drops of water directly on lip stain 16. Alternately, or in addition to this procedure, the user merely moistens their own lips prior to use. Thus, in order for the colored ink of lip stain 16 to be transferred from substrate 12 to the user's lips, lip stain 16 must be moistened. This can occur either by moistening lip stain 16 or by moistening the user's lips, which when the user contacts their lips to lip stain 16, naturally wets the ink of lip stain 16. Thus, the ink of lip stain 16 requires moisture to be transferred properly to the user's lips.

In one embodiment, shown in FIG. 3, the user is able to grasp the outer edges, i.e., the first and second handling areas, of substrate 12 after substrate 12 has been folded, thus keeping their hands free of ink, and then contact their upper lips to the upper exposed surface of 12 and their lower lips to the lower exposed surface of substrate 12. In this fashion, the ink from lip stain 16 is transferred smoothly and evenly to the user's upper and lower lips.

In certain instances, the user may not be able to properly roll their lips on the folded substrate 12 in order to transfer the lip stain 16 to their lips. This could be for a variety of reasons. For example, the user may have had recent dental work and may have trouble pressing their lips down on the lip stain 16. Or, the user may have lip filler which would prevent them from properly rolling their lips entirely on lip stain 16. In these instances, the user need not fold substrate 12, but instead can wet and then pucker their lips and “kiss” the lip stain 16, much like sealing an envelope. This would result in the ink from lip stain 16 be smoothly and properly transferred to the user's lips.

It is often desirable for a user to select lip stains 16 of various colors and shades and to streamline the process of selecting a desired color or shade or lip stain 16, printing the lip stain 16 out on the substrate 12 and repeating the process over and over again. Thus, in another non-limiting embodiment, the present disclosure describes a process where, rather than purchasing lip stain kits of various colors and shades and repurchasing kits when they have run out, users have access to a software application where they can select from a choice of lip stain colors, designs and dimensions, and can download and print out the lip stains 16 on the substrate 12 in their own home or office in order to give the user the ability to select, create and then print out their own selected lip stains 16 without the need to go out and purchase a new kits of lip stain products 10.

In one embodiment, the user signs up for a membership to a service, for example, on a monthly basis. The user is provided access to the service either via a downloadable software application on the user's computer or mobile device, or via access to a website. In one embodiment, as part of the membership, the user will receive a handheld or desktop printer, paper, which serves as the substrate 12 upon which the lip stains 16 are printed, and ink cartridges. In one embodiment, the paper and ink cartridges are reordered automatically. Using the service via the user's computing device, the user may select a lip stain color or colors or shades of their choice. The user also selects the substrate 12 size and the lip stain 16 size. Although “specialized” printer is used herein, it is also within the scope of the present disclosure to allow the user to print out the lip stain product 10 on an ordinary printer.

In one embodiment, in order to print the lip stain 16 on the substrate 12, the user sends a specific number associated with the selected color or shade to the specialized printer that they have been provided. This can be accomplished via any communications substrate, for example, a wireless connection protocol such as Bluetooth®. The present disclosure is not limited by communication established between the user's computing device and the specially designed printer.

In one embodiment, once the number associated with the user's selected color or shade is sent to the specialized printer with the ink cartridges loaded thereon, the specialized printer will print the selected color or shade of lip stain 16 in the selected size on the paper substrate 12. In another embodiment, the user has the ability, via the software application to download a template populated with a variety of colors, shades, sizes, dimensions, etc. Advantageously, this allows the user to select one or more lip stain products 10 of a desired color or shade of lip stain 16 from the template to be printed on the paper substrate on the user's printer. Once the paper 12 is printed out on the user's specialized printer containing the user-selected lip stain 16, the lip stain 16 can be transferred to the user's lips in the manner described above.

In other embodiments, rather than requiring the user to sign up for a membership or subscription and thus be allowed to download the template for multiple uses, a one-time user may be given the opportunity to print out one or more lip stain products 10 from the template without downloading it.

In another embodiment, the lip stain product 10 can be sold and dispensed from a vending machine. For example, the user can select the color/shade/size they would like. In one embodiment this can be done via a touch screen on the vending machine. The user can select and purchase more than one lip stain product 10.

In another embodiment, when the user has signed up for a membership and been granted access to application, the user has the option of subscribing to several categories. For example, customers may subscribe to the “Top Five Influencer Colors of the Year” or “The Most Downloaded Spring Colors.” The user will have the option of picking a specific influencer or makeup artist who they follow. In turn, the influencer/makeup artist will receive a commission from anyone subscribing to the collection.

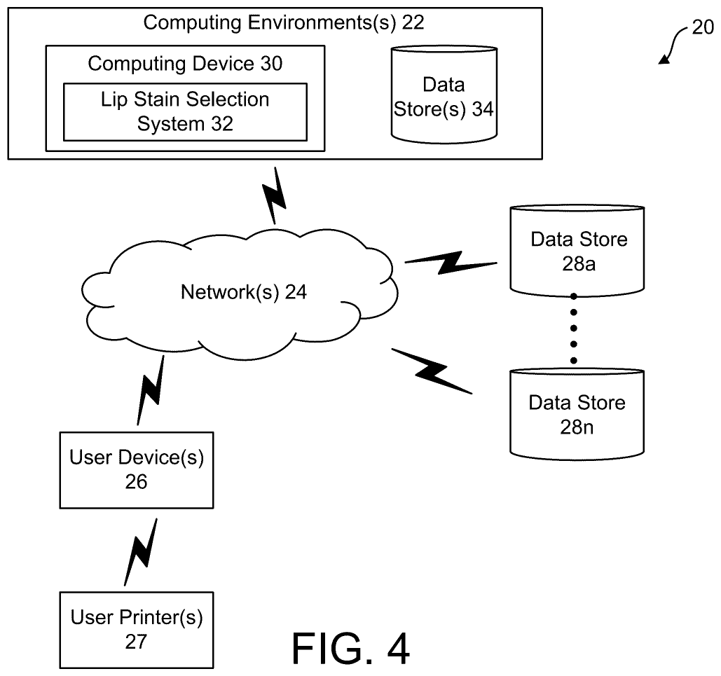

FIG. 4 illustrates a block diagram of an exemplary computer-implemented system 20 that allows users to access and print out selected lip stains 16 on a specialized printer according to some embodiments of the present disclosure. System 20 may include one or more user devices 26 and one or more computing environments 22 that may be in communication with each other via one or more networks 24 (collectively referred to as network 24). Further, system 20 may include one or more data stores 28a-28n (collectively referred to as data store 28) that are in communication with one or more elements of system 20, such as for example, the computing environment 22.

In one or more embodiments, user device 26 may refer to one or more of a computer, laptop, mobile phone, tablet, handheld electronic device, etc. User device 26 is configured to interface and/or communicate with computing environment 22 via network 24, as described herein. User device 26 may include one or more user interfaces (e.g., buttons, touch screen, input devices, etc.) to facilitate a user interacting with user device 26. Thus, as described above, once a user signs up for a membership, the user may be granted access to system 20, which will allow the user to access a large assortment of lip stains 16, which can be printed on a specialized printer 27.

Further, computing environment 22 may include one or more computing devices 30, i.e., servers. In embodiments using multiple computing devices 30, the computing devices 30 may be located in a single installation or may be distributed among many different geographic locations (e.g., cloud computing environment).

Further, computing device 30 may include lip stain selection system 32 and data store(s) 34 for storing one or more lip stains 16 of various colors and shades and/or input data, among other data that may be used by computing environment 22 to allow a user to select, access, and download one or more lip stains 16, for printing out on a specialized computer. Lip stain selection system 32 is configured to provide access to one or more lip stains 16 based on one or more user inputs, as described herein. Further, lip stain selection system 32 may be configured to perform other functionality related to determining input data such as by querying and/or searching one or more data stores 34.

Continuing to refer to FIG. 4, data store 34 of computing environment 14 may be configured to store various information and/or data associated with providing a user with the availability to access, download and print out various lip stains 16, of their choice, as described herein.

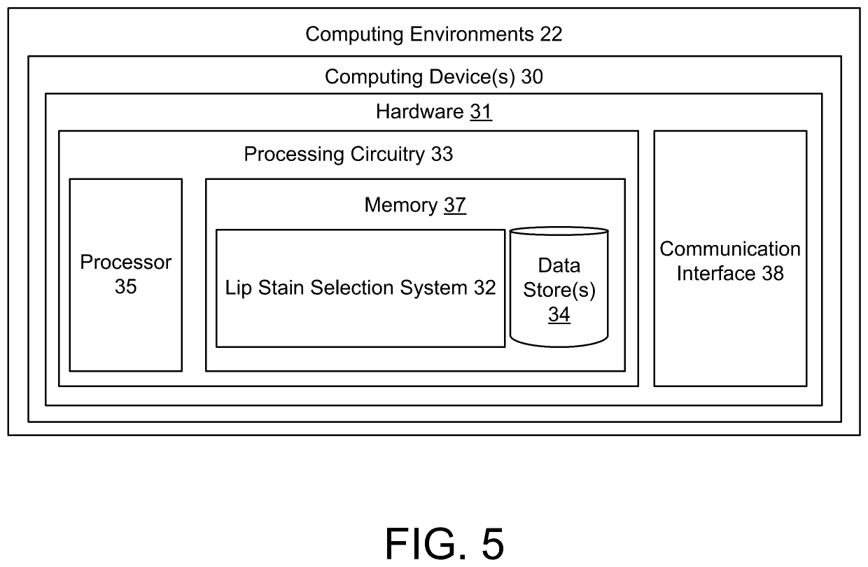

FIG. 5 is a block diagram of an example computing environment 22 of FIG. 4 according to some embodiments of the present disclosure. FIG. 5 is a block diagram illustrating an exemplary computing environment 22 according to various embodiments. As shown, the computing environment 22 may include one or more computing devices 30. As shown, each computing device 30 comprises hardware 31. The hardware 31 may include processing circuitry 33. The processing circuitry 33 may include one or more processors 35 and one or more memories 37. Each processor 35 may include and/or be associated with one or more central processing units, data bases, buffers, and interfaces to facilitate operation. In addition to or instead of a processor 35 and memory 37, the processing circuitry 33 may comprise other types of integrated circuitry that perform various functionality. Processing circuitry 33 may include one or more processors 35, processor cores, FPGAs, ASICs, GPUs, SoCs, or other components configured to execute instructions. The processor 35 may be configured to access (e.g., write to and/or read from) the memory 37, which may comprise any kind of volatile and/or nonvolatile memory, e.g., cache, buffer memory, RAM, ROM, optical memory, and/or EPROM. Further, memory 37 may be embodied in the form of one or more storage devices. The processing circuitry 33 may be configured to perform various functionality described herein. For example, computer instructions may be stored in memory 37 and/or another computer-readable medium that, when executed by processor 35, causes the processor 35 to perform various functionality as described herein.

Hardware 31 may include communication interface 38 facilitating communication between one or more elements in system 20. For example, communication interface 38 may be configured for establishing and maintaining at least a wireless or wired connection with one or more elements of system 20 such as network 24, data store 28, user device 26 etc.

The processing circuitry 33 may be configured to control any of the methods and/or processes described herein and/or to cause such methods, and/or processes to be performed, e.g., in computing environment 22. Processor 35 corresponds to one or more processors 35 for performing computing device 30 functions described herein.

The memory 37 is configured to store data, such as files, data, information, etc. that are described herein. Also stored in the memory 37 and executable by the processor 35 are the lip stain selection system 32. Although FIG. 5 shows the lip stain selection system 32 being in a single computing device 30, the lip stain selection system 32 may execute in multiple computing devices 30 of the computing environment 22. To perform the functionality of the lip stain selection system 32, the memory 37 may include instructions that, when executed by the processor 35 and/or processing circuitry 33, causes the computing device 30 to perform the functionality performed by the lip stain selection system 32 disclosed herein.

Having described aspects of the present disclosure in detail, it will be apparent that modifications and variations are possible without departing from the scope of aspects of the present disclosure as defined in the appended claims. As various changes could be made in the above constructions, products, and methods without departing from the scope of aspects of the present disclosure, it is intended that all matter contained in the above description and shown in the accompanying drawings shall be interpreted as illustrative and not in a limiting sense.

It will be appreciated by persons skilled in the art that the present invention is not limited to what has been particularly shown and described herein above. In addition, unless mention was made above to the contrary, it should be noted that all of the accompanying drawings are not to scale. A variety of modifications and variations are possible in light of the above teachings without departing from the scope and spirit of the invention, which is limited only by the following claims.

1. A lip stain product applicable to and absorbable by a user's lips, the lip stain product comprising:
a substrate having a first side and a second side; and
a lip stain comprised of ink, the lip stain at least one of imprinted and embedded on at least the first side of the substrate, wherein when at least one of the user's lips and the ink is moistened, the lip stain transfers directly to the lips of the user.

2. The lip stain product of claim 1, wherein the lip stain product is produced on a printer using CMYK cartridges filled with ink.

3. The lip stain product of claim 1, wherein the ink is edible.

4. The lip stain product of claim 1, wherein the substrate is soft tissue paper.

5. The lip stain product of claim 1, wherein the substrate is wax paper.

6. The lip stain product of claim 1, wherein the lip stain covers less than 50% of a surface area of the first side of the substrate.

7. The lip stain product of claim 1, wherein the lip stain covers more than 50% of a surface area of the first side of the substrate.

8. The lip stain product of claim 1, wherein the lip stain covers at least 80% of a surface area of the first side of the substrate.

9. The lip stain product of claim 1, wherein the substrate further comprises alignment indicia indicating where the substrate is to be folded prior to transferring the lip stain to the user's lips.

10. The lip stain product of claim 1, wherein the lip stain comprises less than all of the first side of the substrate thus leaving unstained portions of the first side of the substrate.

11. The lip stain product of claim 10, wherein the unstained portions comprise a first handling area and a second handling area allowing the user to grasp each of the first and second handling areas before and while applying the lip stain to the user's lips.

12. The lip stain product of claim 1, wherein the ink comprises water, and at least one of Propylene Glycol, Glycerin, Poly Sorbate 80, and Mono and Di-Glycerides.

13. The lip stain product of claim 1, wherein the ink further comprises at least one of FD&C Red No. 40, FD&C Blue No. 1, FD&C Yellow No. 5, and FD&C Yellow No. 6.

14.-20. (canceled)
